SEQUENCE LISTING (11 sequences)

SEQ ID NO: 1 - protein, 32 aa, Artificial Sequence
  Description of Artificial Sequence Synthetic nesiritide peptide
SPKMVQGSGCFGRKMDRISSSSGLGCKVLRRH

SEQ ID NO: 2 - protein, 39 aa, Artificial Sequence
  Description of Artificial Sequence Synthetic exenatide peptide
HGEGTFTSDLSKQMEEEAVRLFIEWLKDGGPSSGAPPPS

SEQ ID NO: 3 - protein, 36 aa, Artificial Sequence
  Description of Artificial Sequence Synthetic enfuvirtide peptide
YTSLIHSLIEESQNQQEKNEQELLELDKWASLWNWF

SEQ ID NO: 4 - protein, 34 aa, Artificial Sequence
  Description of Artificial Sequence Synthetic teriparatide peptide
SVSEIQLMHNLGKHLNSMERVEWLRKKLQDVHNF

SEQ ID NO: 5 - protein, 31 aa, Artificial Sequence
  Description of Artificial Sequence Synthetic peptide
SNLSTXVLGKLSQELHKLQTYPRTDVGAGTP

SEQ ID NO: 6 - protein, 20 aa, Artificial Sequence
  Description of Artificial Sequence Synthetic bivalirudin peptide
FPRPGGGGNGDFEEIPEEYL

SEQ ID NO: 7 - protein, 33 aa, Artificial Sequence
  Description of Artificial Sequence Synthetic sermorelin peptide
YADAIYADIFTNSYRKVLGQLSARKLLQDIMSR

SEQ ID NO: 8 - protein, 41 aa, Artificial Sequence
  Description of Artificial Sequence Synthetic corticorelin peptide
SQEPPISLDLTFHLLREVLEMTKADQLAQQAHSNRKLLDIA

SEQ ID NO: 9 - protein, 28 aa, Artificial Sequence
  Description of Artificial Sequence Synthetic thymosin alpha 1 peptide
SDAAVDTSSEITTKDLKEKKEVVEEAEN

SEQ ID NO: 10 - protein, 27 aa, Artificial Sequence
  Description of Artificial Sequence Synthetic secretin peptide
HSDGTFTSELSRLRDSARLQRLLQGLV

SEQ ID NO: 11 - protein, 37 aa, Artificial Sequence
  Description of Artificial Sequence Synthetic pramlintide peptide
KCNTATCATQRLANFLVHSSNNFGPILPPTNVGSNTY

(19) United States
(12) Patent Application Publication
(10) Pub. No.: US 2008/0287650 A1
(43) Pub. Date: Nov. 20, 2008

(54) High purity peptides

(76) Inventors: Avi Tovi, Zurit (IL); Chaim Eidelman, Oshrat (IL); Shimon Shushan, Karmiel (IL); Shai Elster, Haifa (IL); Alon Hagi, Kibbutz Yehiam (IL); Alexander Ivchenko, Keryat Ata (IL); Gabriel-Marcus Butilca, Karmiel (IL); Gil Zaoui, Keryat Bialik (IL); Eleonora Alterman, Haifa (IL); Leah Bar-Oz, Kiryat Bialik (IL)

Correspondence Address: KENYON & KENYON LLP, One Broadway, New York, NY, 10004, US

(21) Appl. No.: 12/074,454

(22) Filed: Mar. 3, 2008

Related U.S. Application Data
(60) Provisional application No. 60/904,512, filed on Mar. 1, 2007.
(60) Provisional application No. 60/995,652, filed on Sep. 26, 2007.
(60) Provisional application No. 60/997,285, filed on Oct. 1, 2007.

(51) Int. Cl. C07K 1/00; C07K 1/04; C07K 7/08; C07K 7/64; C07K 14/00

(52) U.S. Cl. 530/317; 530/335; 530/334; 530/324; 530/326

(57) ABSTRACT

The invention relates to methods for the preparation of highly purified peptides. The peptides are prepared in high optical purity of at least about 98.5%, and preferably at least about 99%. Specifically, Nesiritide (SEQ. ID NO. 1) having a purity of at least 99% as measured by HPLC and containing about 0.05% to about 0.5% [D-His]-Nesiritide (SEQ. ID NO. 1) as measured by chiral GC/MS.

### CROSS-REFERENCE TO RELATED APPLICATIONS

This application claims the benefit of U.S. provisional application Ser. Nos. 60/904,512, filed Mar. 1, 2007, 60/995,652, filed Sep. 26, 2007, and 60/997,285, Oct. 1, 2007, hereby incorporated by reference.

### FIELD OF THE INVENTION

The invention encompasses processes for the preparation and purification of peptides.

### BACKGROUND OF THE INVENTION

Peptide-based drugs provide therapies for a broad range of disorders. However, the rate of peptide drug development is slowed by obstacles encountered during peptide synthesis.

As solid phase peptide synthesis techniques improved the rate at which a peptide could be synthesized, purification became the limiting factor in the production of high-quality peptides. Purification techniques improved with the introduction of reversed phase HPLC (high performance liquid chromatography). However, purification and separation problems remain for some structurally similar peptides that have, for example, side-chain modifications, amino acid deletions or additions, diastereomeric (racemized) peptides, truncated peptides, reaction by-products, deamidation peptides, by-products generated by incomplete deprotection of amino acid side chain protecting groups, oxidized peptides, disulfide exchange products, oligomers and/or aggregates or toxic reagents and solvents used in synthesis. It is desirable to eliminate these impurities so a particular compound's properties may be analyzed more accurately and because biological activity generally resides in one enantiomer. B. Waldeck, Chirality, 5(5): 350-355 (1992).

A particularly problematic side reaction in peptide chemistry is racemization, which results in partial loss of chiral purity among amino acid residues. All amino acids (except glycine) have a chiral α-carbon. Thus, securing a target peptide in homogeneous form generally requires enantiomerically pure amino acid starting materials as well as conservation of chiral homogeneity throughout the various operations of peptide synthesis. If chirality is not preserved throughout peptide synthesis, a mixture of diastereoisomers will be obtained instead of a single chiral product. Separation of the desired peptide from a multitude of similar (racemic) compounds can be complex and labor-intensive. Thus, strategies for peptide synthesis are primarily motivated by conservation of chiral homogeneity. M. Bodanzky, PEPTIDE CHEMISTRY, A PRACTICAL TEXTBOOK (Springer-Verlag 1988).

The degree of racemization that occurs during peptide synthesis is affected by various parameters including activation strategy, reaction conditions, protection strategy and the nature of the amino acid residue. It is well known that amino acids have varying degrees of sensitivity to racemization. Histidine, for example, is particularly prone to racemization due to its chemical structure, which contains an imidazole functional group. K. Hofmann et al., JACS 80, 1486 (1958); J. Syrier et al., Recl. Trav. Chim. Pay-Bas 93, 117 (1974). It has been shown that racemization can be decreased by blocking the N(π)-nitrogen. J. H. Jones et al., J. Chem. Soc., Chem. Commun., 472 (1978); J. H. Jones et al., J. Chem. Soc., Perkin Trans. 1, 2261 (1979). However, these protecting building blocks are not available on a commercial scale and are difficult to incorporate into production processes. Other commercially available protected histidine derivatives provide much lower protection against racemization. Use of urethane protected amino acids (such as Boc, Cbz, Fmoc), for example, largely solved the racemization problem during sequential peptide syntheses using histidine.

However, racemization remains a problem when a peptide sequence ends with a free carboxylic group and contains a histidine residue at the C-terminal of the peptide (which is usually a starting point of the peptide synthesis). Novabiochem Catalog 2006/2007, Synthesis Notes. In such a case, synthesis on a solid support can be problematic because loading the protected histidine on a support resin would result in partial loss of chiral purity. Id. Synthesis of Nesiritide (SEQ. ID NO. 1), which has a carboxyl-terminal histidine, on a solid support was described by Akaji et al., JACS, 114(11): 4137 (1992). Akaji et al. described preparation of Nesiritide (SEQ. ID NO. 1) on Wang resin using Fmoc-His(Bum)-OH as the starting material. According to the literature, Bum protection could reduce (but not completely avoid) the degree of stereomutation of the chiral center, yet the actual amount of racemization was not determined in this work. See, e.g., Mergler, et al., J. Peptide Science, 7(9): 502-510 (2001). Furthermore, Fmoc-His(Bum)-OH is not commercially available and thus cannot serve as a starting material for industrial processes. According to the literature, the method of choice in the case of racemization-prone amino acids should be use of trityl-based resins. Novabiochem Catalog 2006/2007, Synthesis Notes, page 2:18. However, synthesis of Nesiritide (SEQ. ID NO. 1) on 2-chlorotrityl-chloride resin can result in peptides with a high amount from about 1% to about 13% of the unnatural conformation of histidine, [D-His] (inventors' unpublished results).

Although many diastereomeric impurities that occur during peptide synthesis can be removed by preparative HPLC, it is difficult to separate some diastereomers such as [D-His]-Nesiritide (SEQ. ID NO. 1) by HPLC technique alone. Thus, in some cases it may be advantageous to synthesize crude peptides with high chiral purity before HPLC treatment. The invention seeks to solve the problems encountered by the prior art by providing a synthetic strategy and purification technique that strategically deprotects a polypeptide in a manner that precludes the failures described above.

### SUMMARY OF THE INVENTION

The present invention encompasses processes for preparing peptides of high purity. In one embodiment, the invention encompasses a process for preparing a peptide of high purity comprising: (a) providing a fully protected peptide having at least one acid labile protecting group and at least one orthogonal protecting group; (b) deprotecting the acid labile protecting groups from the fully protected peptide with an acidic composition yielding a semi-protected peptide; (c) purifying the semi-protected peptide by HPLC; (d) deprotecting the orthogonal protecting groups from the semi-protected peptide with a deprotecting agent yielding a fully deprotected peptide; and (e) purifying the fully deprotected peptide by HPLC, wherein if the fully protected peptide contains two or more thiol-containing residues, then all the thiol-containing residues are protected by the acid labile protecting groups. The process optionally includes at least one of the following steps: neutralizing excess deprotecting agent, drying the fully deprotected peptide, performing a counter-ion exchange of the fully deprotected peptide with a suitable ion, adding a histidine residue onto the carboxyl terminal of a fully protected peptide, or cyclizing a fully deprotected peptide.

In another embodiment, the peptide produced by the process is preferably Nesiritide (SEQ. ID NO. 1), Teriparatide (SEQ. ID NO. 4), Bivalirudin (SEQ. ID NO. 6), Exenatide (SEQ. ID NO. 2), Sermorelin (SEQ. ID NO. 7), Corticorelin (SEQ. ID NO. 8), Enfuvirtide (SEQ. ID NO. 3), Thymosin alpha 1 (SEQ. ID NO. 9), Secretin (SEQ. ID NO. 10), Pramlintide (SEQ. ID NO. 11) or Elcatonin (SEQ. ID NO. 5).

In another embodiment, the invention encompasses producing a peptide having a purity of at least about 97.5% by HPLC and preferably, having a purity of at least about 98.5%. Most preferably, at least about 99% by HPLC.

In another embodiment, the present invention encompasses Nesiritide (SEQ. ID NO. 1) having a purity of at least 99% as measured by HPLC and containing about 0.05% to about 0.5% [D-His]-Nesiritide (SEQ. ID NO. 1) as measured by chiral GC/MS.

In another embodiment, the present invention encompasses a process for preparing Nesiritide (SEQ. ID NO. 1) comprising: (a) providing a fully protected peptide attached to a highly acid sensitive resin having the formula X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-O-Resin (SEQ. ID NO. 1), wherein X is an orthogonal or acid-labile protecting group, U is an orthogonal or acid-labile protecting group on a cysteine residue and Y is an acid-labile protecting group; (b) reacting the fully protected peptide with a weak acidic composition to cleave the fully protected peptide from the resin, providing the fully protected peptide X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-OH (SEQ. ID NO. 1) in solution; (c) coupling H-His(X)-O(Z) to the fully protected peptide of step (b) to produce X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-His(X)-O(Z) (SEQ. ID NO. 1), wherein Z is a carboxyl-terminal histidine protecting group which is either an orthogonal or acid-labile protecting group, see definition below for “carboxyl-terminal histidine protecting group”; (d) isolating the fully protected peptide by either evaporation of a solvent or by precipitating the peptide using a suitable co-solvent; (e) deprotecting the acid labile protecting groups in the fully protected peptide by treatment with an acidic composition to produce a semi-protected peptide, X-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His(X)-OZ (SEQ. ID NO. 1) or a non-protected linear peptide H-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His-OH (SEQ. ID NO. 1); (f) purifying the semi-protected peptide by preparative HPLC; (g) if necessary, deprotecting the orthogonal protecting groups X, U and Z from the semi-protected peptide to provide a fully deprotected peptide; (h) purifying the fully deprotected peptide by preparative HPLC; (i) cyclizing the non-protected linear peptide to provide a cyclic peptide; (j) purifying the cyclic peptide by preparative HPLC; (k) exchanging the counter ion of the fully deprotected peptide to citrate; and (l) drying the fully deprotected peptide to provide Nesiritide (SEQ. ID NO. 1) in solid powder, wherein, the orthogonal protecting group remained on the semi-protected cysteine residues can be deprotected from the peptide during the cyclization step using iodine. Preferably, the highly acid sensitive resin is 2-chlorotrityl-chloride. In this specific case of nesiritide preparation, the purity of the peptide in regards to D-His content (chiral purity) is not dependent on the HPLC purification but on the synthetic route alone. Therefore, it does not matter if the peptide is purified via its fully unprotected form or as its semi-protected form first (in any case D-His impurity cannot be removed by HPLC purification). However, purification of the semi-protected peptide can aid in purification of other impurities thus providing higher HPLC purity of the peptide.

In yet another embodiment, the present invention encompasses a peptide-resin conjugate of formula 1:

A-B-Resin

wherein A=X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-O-, X and U are an orthogonal or acid-labile protecting groups, Y is an acid-labile protecting group; B is 2-chlorotritylchloride, and Resin is a solid matrix attached to the peptide. Preferably, the protecting group (X) is 9-fluorenylmethyloxycarbonyl (“Fmoc”) or t-butyloxycarbonyl (“Boc”) and the protecting group (U) is acetamidomethyl (“Acm”) or trityl (“Trt”).

### DETAILED DESCRIPTION OF THE INVENTION

The invention encompasses processes for preparing peptides of high purity. More specifically, the invention encompasses a process for preparing polypeptides wherein at least one orthogonal protecting group attached to at least one amino terminal residue, carboxyl terminal residue, or amino group that is not cleaved or deprotected from the protected residue under conditions required for cleavage of other acid labile protecting groups, and wherein if the fully protected peptide contains two or more thiol-containing residues, then all thiol-containing residues are protected by the acid labile protecting groups.

A peptide protected by at least one orthogonal protecting group, but not by an acid labile protecting group, is referred to as a semi-protected peptide. A peptide protected by at least one orthogonal protecting group and by at least one acid labile protecting group is referred to as a fully protected peptide. A peptide from which all orthogonal protecting groups and acid labile protecting groups have been cleaved is referred to as a fully deprotected peptide.

As used herein, the term “acid labile protecting group” refers to a protecting group on an amino acid residue and that is cleaved from the peptide under a different set of conditions than the orthogonal protecting group, as explained below.

As used herein, the term “orthogonal protecting group” refers to a protecting group on at least one amino terminal residue, carboxyl terminal residue, or amino group and which has not been cleaved or deprotected from a protected residue under conditions required for cleavage of acid labile protecting groups.

As used herein, the term “acidic composition” refers to a composition capable of removing an acid labile protecting group.

As used herein, the term “deprotecting agent” refers to an agent capable of removing an orthogonal protecting group.

The process for preparing a peptide of high purity comprises (a) providing a fully protected peptide having at least one acid labile protecting group and at least one orthogonal protecting group; (b) deprotecting the acid labile protecting groups from the fully protected peptide with an acidic composition yielding a semi-protected peptide; (c) purifying the semi-protected peptide by HPLC; (d) deprotecting the orthogonal protecting groups from the semi-protected peptide with a deprotecting agent yielding a fully deprotected peptide; and (e) purifying the fully deprotected peptide by HPLC, wherein if the fully protected peptide contains two or more thiol-containing residues, then all the thiol-containing residues are protected by the acid labile protecting groups.

In most cases, after treatment of the fully protected peptide with an acidic composition, the resultant semi-protected peptide chain is not fully deprotected, it still carries at least one orthogonal protecting group attached to at least one of the amino terminal residue, carboxyl terminal residue, or amino group. After treatment of the semi-protected peptide with a deprotecting agent, the resultant peptide chain will then be fully deprotected. In short, this is achieved via two deprotecting steps: one that first removes all acid labile protecting groups and then a second step that removes all orthogonal protecting groups.

Peptides that may be produced by this process include, but are not limited to, Nesiritide (SEQ. ID NO. 1), Teriparatide (SEQ. ID NO. 4), Bivalirudin (SEQ. ID NO. 6), Exenatide (SEQ. ID NO. 2), Sermorelin (SEQ. ID NO. 7), Corticorelin (SEQ. ID NO. 8), Enfuvirtide (SEQ. ID NO. 3), Thymosin alpha 1 (SEQ. ID NO. 9), Secretin (SEQ. ID NO. 10), Pramlintide (SEQ. ID NO. 11) or Elcatonin (SEQ. ID NO. 5).

Preparation of the fully protected peptide may be performed by any known method in the art for peptide synthesis, such as on a solid support or in solution, among others. Such methods can be found in Paul Lloyd-Williams, et al., CHEMICAL APPROACHES TO THE SYNTHESIS OF PEPTIDES AND PROTEINS, (C. W. Rees, ed. CRC Press 1997) hereby incorporated by reference.

When synthesizing the peptide on a solid support, suitable resins for use in the process include, but are not limited to 2-chlorotrityl-chloride (“CTC”) resin, Rink amide resin or Wang resin. Suitable coupling agents include, but are not limited to 2-(1H-benzotriazole-1-yl)-1,1,3,3-tetramethyluronium tetrafluoroborate (“TBTU”), O-benzotriazole-N,N,N′,N′-tetramethyl-uronium-hexafluoro-phosphate (“HBTU”), benzotriazol-1-yl-oxytripyrrolidinophosphonium hexafluorophosphate (“PyBOP”), N,N′-dicyclohexylcarbodiimide (“DCC”), or N,N′-diisopropylcarbodiimide (“DIC”).

One of ordinary skill in the art with little or no experimentation can easily determine suitable solvents used in the washing steps. Preferably, suitable solvents for use in the washing steps of the process include, but are not limited to, N,N-dimethylformamide (“DMF”), dichloromethane (“DCM”), methanol (“MeOH”), or isopropanol (“IPA”). When a peptide is synthesized on a resin, the resin may be washed with at least one solvent to remove any soluble compounds from the resin after addition of each new amino acid. Suitable solvents are known in the art and include, but are not limited to N,N-dimethylformamide (“DMF”), dichloromethane (“DCM”), isopropyl alcohol (“IPA”), N,N-dimethylacetamide (“DMA”) or N-methylpyrrolidone (“NMP”).

Optionally, each newly added amino acid is protected with an orthogonal protecting group and the orthogonal protecting group is optionally cleaved before coupling with the next amino acid. This process may be repeated until all desired amino acids have been added. When the peptide is synthesized on a solid phase, such as on a resin, it is preferably synthesized using the Fmoc method. The acidic composition used for deprotection of acid sensitive protecting groups may also cleave the resulting semi-protected peptide from the resin. Alternatively, acid-labile protecting groups may be removed without removing the peptide from the resin. In this case, in the second deprotection step, the peptide and the orthogonal protecting groups may be cleaved from the resin either sequentially or simultaneously.

Optionally, the fully protected peptide is first cleaved from the resin using a weak acidic composition containing 2% trifluoroacetic acid (“TFA”) by volume in dichloromethane (“DCM”). The weak acidic compositions may include, but are not limited to, 0.5% to 5% TFA in DCM, acetic acid (“AcOH”)/trifluoroethanol (“TFE”)/DCM in a ratio of about 2:2:6, or 30% hexafluoroisopropanol (“HFIP”) in DCM.

Once the fully protected peptide has been synthesized, it will undergo a first deprotection step wherein acid labile protecting groups are removed using an acidic composition to yield a semi-protected peptide. Preferably, acid labile protecting groups are removed by washing the fully protected peptide with an acidic composition. One of ordinary skill in the art can determine the appropriate conditions for this deprotection step. See M. Bodanzky, PEPTIDE CHEMISTRY, A PRACTICAL TEXTBOOK (Springer-Verlag 1988).

Acid labile protecting groups preferably include, but are not limited to t-butyl ester (“OtBu”), trityl (“Trt”), t-butyloxycarbonyl (“Boc”), 2,2,4,6,7-pentamethyldihydrobenzofuran-5-sulfonyl (“Pbf”) or t-butyl (“tBu”).

Acidic compositions may also include at least one scavenger reagent. Suitable scavenger reagents include, but are not limited to, organosilanes, phenol derivatives or thiol-containing compounds, such as, triisopropylsilane (“TIS”), 1,2-ethanedithiol (“EDT”), water, phenol, m-cresol, thioanisole, dithioerythritol (“DTE”) or dodecylmercaptane (“DDM”). Typically, the acidic material in the acidic composition is an acid and scavenger reagents. Suitable acids include, but are not limited to, trifluoroacetic acid (“TFA”), hydrofluoric acid (“HF”), trifluoromethanesulfonic acid (“TFMSA”) or hydrobromic acid (“HBr”)/AcOH.

Preferably, the acidic material is present in an amount of about 50% to about 99% by volume of the acidic composition, and the scavenger reagents are present in an amount of about 1% to about 50% by volume of the acidic composition. More preferably, the acidic material is TFA and the acidic composition has at least about 80% TFA. Most preferably, the acidic composition has approximately 95% TFA, 2.5% TIS and 2.5% EDT. The relative ratio of the acid to scavenger to water may be from about 85% to about 99% acid material, from about 0.1% to about 15% scavenger, and from about 0.1% to about 15% water by volume. The amounts of the acidic material, scavenger reagents and water for each acidic composition may vary depending on the peptide being synthesized. In one example embodiment, the acidic composition contains about 95% TFA, about 2.5% EDT, and about 2.5% water.

After the first deprotection step, the semi-protected peptide undergoes a coarse purification using HPLC. As used herein, the term “coarse purification” refers to chromatographic purification of the semi-protected peptide after the acid-labile protecting groups are removed by treatment under acidic conditions, as explained above. Coarse purification by preparative HPLC produces the semi-protected peptide having a purity of at least about 95% by area HPLC or by weight using a standard in the HPLC, preferably having a purity of at least about 98.5%, and most preferably having a purity of at least about 99% as determined by HPLC, and at a concentration of about 0.1 g/L to about 100 g/L. Preferably, the peptide has a concentration of 0.1 g/L to about 10 g/L.

The coarse purification of the semi-protected peptide by preparative HPLC includes running a mobile phase comprising the semi-protected peptide, organic solvents and an aqueous buffer through an HPLC column packed with a stationary phase such as C-8 or C-18 silica. Suitable organic solvents are known in the art and include, but not limited to, acetonitrile (“ACN”), methanol, propanol or tetrahydrofuran (“THF”). On average, the retention time for a semi-protected peptide is longer than the retention time of a non-protected peptide due to presence of a hydrophobic protecting group. The same or slightly modified preparative HPLC conditions may be used when purifying semi-protected and non-protected peptides because the difference in retention time between semi-protected and non-protected peptides is not large enough to require completely different separation conditions. As will be understood by those skilled in the art, the parameters of the gradient elution program and buffer may be adjusted if the difference in retention time between semi-protected and non-protected peptides is insufficient to achieve separation. In any case the conditions should be adjusted in such a way that the purified material elutes in reasonable (practical) time from the column. However the issue is not separation between semi-protected and non-protected peptides but purification of each one of them from other impurities. The difference in retention time between semi-protected and non-protected peptides must be sufficient enough to achieve good purification of semi-protected peptide from other impurities that elute at about the same retention time as the non-protected peptide.

Following coarse purification, the purified semi-protected peptide is fully deprotected using a deprotecting agent to cleave the orthogonal protecting groups. Typically, orthogonal protecting groups are removed by reacting the semi-protected peptide with a deprotecting agent. The appropriate conditions for deprotection of orthogonal protecting groups in peptide syntheses are readily ascertainable by one of ordinary skill in the art with little or no experimentation.

Orthogonal protecting groups may be selectively attached to residues of amino acids. Orthogonal protecting groups include, but are not limited to, 9-fluorenylmethyl-oxycarbonyl (“Fmoc”), 9-fluorenymethyl ester (“OFm”), benzyloxycarbonyl (“CBZ”), benzyl (“Bzl”), benzyl ester (“OBzl”), α,α-dimethyl-3,5-dimethoxybenzyloxylcarbonyl (“Ddz”), 1-(4,4-dimethyl-2,6-dioxocyclohex-1-ylidene)ethyl (“Dde”), 2,4-dinitrophenyl (“DNP”), N^{α}-2-(4-Nitrophenylsulfonyl)ethoxycarbonyl (“NSC”), allyloxycarbonyl (“aloc”) or acetamidomethyl (“Acm”). When an amino group is protected, the orthogonal protecting group is preferably Fmoc or Boc. When an amino terminal group is protected, the orthogonal protecting group is preferably Fmoc or CBZ and more preferably, it is Fmoc. When a carboxyl terminal group is protected, the orthogonal protecting group is preferably OFm or OBzl. When a lysine residue is protected, the orthogonal protecting group is preferably CBZ. When a histidine residue at the amino terminal is protected, the orthogonal protecting group is preferably Fmoc or DNP.

Fmoc is a preferred orthogonal protecting group on the α-amino group of the amino terminal group in amino acid. When Fmoc protecting group of the last amino acid is not removed prior to cleavage from the resin, acidolytic cleavage of the peptide will yield a peptide sequence carrying at least one Fmoc group.

Deprotecting agents may include, but are not limited to, piperidine, 1,8-diazobicyclo[5.4.0]undec-7-ene (“DBU”), p-dimethylaminopyridine, triethylamine (“TEA”), HBr/AcOH, H₂/Pd/C, hydrazine, hydrofluoric acid, or trifluoromethanesulfonic acid. Preferably, the deprotecting agent is piperidine. When the orthogonal protecting group is Fmoc or DNP, the deprotecting agent is preferably piperidine. When the orthogonal protecting group is CBZ, the deprotecting agent is preferably HBr/AcOH. Typically, adding an equivalent amount of deprotecting agent to the semi-protected peptide will result in deprotection of its orthogonal protecting groups.

Once the peptide is fully deprotected, it undergoes a second purification step. An evaluation of the chromatographic profile at this stage shows that some of the impurities that were previously eluted close to the main (product) peak are now clearly distinct from the product peak. Thus, the fully deprotected peptide can be easily purified a second time by methods such as HPLC or other known methods to obtain a peptide of high optical purity.

The step for purifying the fully deprotected peptide by preparative HPLC includes running a mobile phase comprising the fully deprotected peptide, organic solvents and aqueous buffer through an HPLC column packed with a stationary phase such as C-8 or C-18 silica-based resin. Suitable organic solvents are known in the art and include, but not limited to, acetonitrile (“ACN”), methanol, propanol or tetrahydrofuran (“THF”). The mobile phase may be, for example, 0.02%-0.05% (v/v) TFA in water or acetonitrile. Gradient elution programs employ two solvents: an aqueous phase and an organic phase. The organic phase may be acetonitrile. Preferably, the preparative HPLC technique is reverse phase (RP) HPLC, during which peptide is eluted by increasing the percentage of organic solvent. The organic phase may vary from about 10% to about 40% as a function of time. Ultraviolet (UV) detection may occur at various wavelengths including, but not limited to, 214 nm or 220 nm. Although elution times vary, generally, semi-protected peptides elute slower than fully deprotected peptides because the semi-protected peptides contain a hydrophobic protecting group. One ordinary skilled in the art would be able to customize preparative HPLC parameters depending on the particular peptide being purified. See, e.g., G. Grant, SYNTHETIC PEPTIDES: A USER'S GUIDE 223-227 (Oxford University Press 1992) hereby incorporated by reference.

By these methods, a highly purified product is easily obtainable in high yield, whereas the conventional method undergoes several purification cycles that consumes large volumes of solvents, requires long operation time and the final product obtained has a lower purity and yield. Although the peptides are preferably purified by chromatography, other means of purification known in the art may be used including, but not limited to ion exchange, crystallization, or extraction.

Optionally, a fully deprotected peptide can be cyclized by thiol oxidation using an oxidizing agent such as iodine. The resulting cyclic peptide can optionally be purified using any suitable methods known to those skilled in the art to obtain a cyclic peptide of high purity. See, e.g., G. Grant, SYNTHETIC PEPTIDES: A USER'S GUIDE 223-227 (Oxford University Press 1992) hereby incorporated by reference. As used herein, the term “cyclic peptide” refers to a peptide containing at least two thiol-containing residues connected by a disulfide bridge. Optionally, if excess oxidizing agent is present it can be neutralized prior to purification.

Optionally, a fully deprotected peptide may be further purified by counter ion exchange using a suitable ion. For example, counter ions may be exchanged to citrate, acetate, pamoate, trifluoroacetate, or hydrochloride. U.S. Publication No. 2006/0148699, hereby incorporated by reference, describes suitable counter-ion exchange methods, including loading a peptide onto a RP-HPLC column, washing the column with an aqueous solution of a pharmaceutically acceptable counterion salt, and eluting the peptide from the column with a solvent mixture of a organic solvent and an acid of the pharmaceutically acceptable counterion.

Optionally, a histidine (His) residue may be added onto the carboxyl terminal, a protected His residue in the form of H-His(X)-OZ, may be coupled to a peptide fragment to avoid racemization. Z is carboxyl-terminal histidine protecting group and X is the same or a different protecting group. As used herein, the term “carboxyl-terminal histidine protecting group” is any protecting group that may be removed from the carboxyl-terminal histidine without damaging the peptide fragment. During synthesis, the carboxyl-terminal histidine protecting group is removed after or in parallel to removal of acid labile protecting groups. However, it may also be removed prior to removing all orthogonal protecting groups. The peptide fragment may be a protected peptide. Preferably, the H-His(X)-OZ is H-His(Trt)-OtBu. When histidine is coupled to the carboxyl terminal of a peptide fragment as H-His(Trt)-OtBu, the deprotecting agent is preferably TFA/TIS/EDT.

Optionally, the process for preparing peptides of high purity further comprises neutralizing excess deprotecting agent prior to purification. When the deprotecting agent is basic, neutralization may be accomplished using mineral acids or organic acids. Suitable mineral acids include, but are not limited to, phosphoric acid, hydrochloric acid, sulfuric acid or nitric acid. Suitable organic acids include, but are not limited to, acetic acid or trifluoroacetic acid. Preferably, the neutralization is accomplished using phosphoric acid.

Optionally, the process for preparing peptides of high purity further includes drying the peptide. The drying step may be performed using methods commonly known to the skilled artisan including, but not limited to, spray drying or lyophilization to produce a powder. For example, lyophilization is performed as described in Pharmaceutical Research, 21(2), February 2004 hereby incorporated by reference. Optionally, the fully deprotected, purified peptide solution is concentrated prior to drying.

The process described thus far provides peptides of high purity. As used herein, the term “high purity” refers to a composition comprising at least about 98.5% as determined by HPLC or another analytical method, and preferably at least about 99% by HPLC or another analytical method (for example by chiral gas chromatography with mass spectrometry (GC/MS)). The process described above generally results in semi-protected peptides having greater than 95% purity and fully-deprotected peptides having greater than 97.5%, greater than 98.5% or greater than 99.5% purity by HPLC.

Nesiritide (SEQ. ID NO. 1) can be synthesized by the process described above with a purity of at least about 97.5% as measured by HPLC and containing [D-His]-Nesiritide (SEQ. ID NO. 1)<1.0% (as measured by chiral GC/MS). Preferably, Nesiritide (SEQ. ID NO. 1) has a purity of at least about 98.5% as measured by HPLC, more preferably, it has a purity of at least about 99.0% as measured by HPLC and containing about 0.05% to about 0.5% [D-His]-Nesiritide (SEQ. ID NO. 1) as determined by chiral GC/MS. Also Nesiritide (SEQ. ID NO. 1) can be made containing [D-His]-Nesiritide (SEQ. ID NO. 1)<1.0% (as measured by chiral GC/MS) and preferably containing about 0.05% to about 0.5% [D-His]-Nesiritide (SEQ. ID NO. 1) as determined by chiral GC/MS.

Teriparatide (SEQ. ID NO. 4) can be synthesized by the process described above with a purity of at least about 97.5% by HPLC. Preferably, Teriparatide (SEQ. ID NO. 4) has a purity of at least about 98.5% by HPLC.

Bivalirudin (SEQ. ID NO. 6) can be synthesized by the process described above with a purity of at least about 98.5% as measured by HPLC. Preferably, Bivalirudin (SEQ. ID NO. 6) has a purity of at least about 99% as measured by HPLC and containing not more than 0.5% [Asp⁹-Bivalirudin] (SEQ. ID NO. 6), not more than 0.5% [+Gly]-Bivalirudin (SEQ. ID NO. 6) and not more than 0.5% of any other impurity. Also, Bivalirudin (SEQ. ID NO. 6) can be synthesized by the process described above containing not more than 0.5% [Asp⁹-Bivalirudin] (SEQ. ID NO. 6), preferably not more than 0.5% [+Gly]-Bivalirudin (SEQ. ID NO. 6) and more preferably, not more than 0.5% of any other impurity.

Exenatide (SEQ. ID NO. 2) can be synthesized by the process described above with a purity of at least about 97.5% as measured by HPLC. Preferably, Exenatide (SEQ. ID NO. 2) has a purity of at least about 98.5% as measured by HPLC.

Sermorelin (SEQ. ID NO. 7) can be synthesized by the process described above with a purity of at least about 98.5% as measured by HPLC. Preferably, Sermorelin (SEQ. ID NO. 7) has a purity of at least about 99% as measured by HPLC.

Corticorelin (SEQ. ID NO. 8) can be synthesized by the process described above with a purity of at least about 98.5% as measured by HPLC. Preferably, Corticorelin (SEQ. ID NO. 8) has a purity of at least about 99% as measured by HPLC.

Enfuvirtide (SEQ. ID NO. 3) can be synthesized by the process described above with a purity of at least about 98.5% as measured by HPLC. Preferably, Enfuvirtide (SEQ. ID NO. 3) may have a purity of at least about 99% as measured by HPLC.

Elcatonin (SEQ. ID NO. 5) can be synthesized by the process described above with a purity of at least about 98.5% as measured by HPLC. Preferably, Elcatonin may have a purity of at least about 99% as measured by HPLC.

Thymosin alpha 1 (SEQ. ID NO. 9) can be synthesized by the process described above with a purity of at least about 97.5% as measured by HPLC. Preferably, Thymosin alpha 1 (SEQ. ID NO. 9) has a purity of at least about 98.5% as measured by HPLC.

Secretin (SEQ. ID NO. 10) can be synthesized by the process described above with having a purity of at least about 98.5% as measured by HPLC. Preferably, Secretin (SEQ. ID NO. 10) has a purity of at least about 99% as measured by HPLC.

Pramlintide (SEQ. ID NO. 11) can be synthesized by the process described above with a purity of at least about 98.5% as measured by HPLC. Preferably, Pramlintide (SEQ. ID NO. 11) may have a purity of at least about 99% as measured by HPLC.

The following paragraphs exemplify particular embodiments of the above-described process. These examples are not meant to be limiting but merely be illustrative of the process.

In an exemplary embodiment demonstrating coupling of a histidine residue to the carboxyl terminal of a peptide, Nesiritide (SEQ. ID NO. 1) is produced by providing a fully protected peptide attached to a resin having the formula Fmoc-Ser(Y)-Pro-Lys(Y)-Met-Val-Gln(Y)-Gly-Ser(Y)-Gly-Cys(Y)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(Y)-Lys(Y)-Val-Leu-Arg(Y)-Arg(Y)-O-Resin (SEQ. ID NO. 1), wherein Fmoc is an orthogonal protecting group and Y is an acid-labile protecting group. The fully protected peptide-resin is reacted with a weak acidic composition to cleave the peptide from the resin, yielding the fully protected peptide Fmoc-Ser(Y)-Pro-Lys(Y)-Met-Val-Gln(Y)-Gly-Ser(Y)-Gly-Cys(Y)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(Y)-Lys(Y)-Val-Leu-Arg(Y)-Arg(Y)-OH (SEQ. ID NO. 1) in solution. The fully protected peptide is then coupled with H-His(X)-O(Z) in solution to produce Fmoc-Ser(Y)-Pro-Lys(Y)-Met-Val-Gln(Y)-Gly-Ser(Y)-Gly-Cys(Y)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(Y)-Lys(Y)-Val-Leu-Arg(Y)-Arg(Y)-His(X)-O(Z) (SEQ. ID NO. 1), wherein X is an acid labile or orthogonal protecting group and Z is a carboxyl-terminal histidine protecting group. That peptide is isolated by evaporating the solvent from the solution or by precipitating the fully protected peptide using a suitable co-solvent. The acid labile protecting groups are removed from the fully protected peptide by treatment with an acidic composition to produce a semi-protected peptide, Fmoc-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His(X)-OZ (SEQ. ID NO. 1), which is then purified using HPLC. If X and Z protecting groups on histidine residues are not acid labile, they are removed by treating the semi-protected peptide with a deprotecting agent to remove the orthogonal protecting groups on the amino and carboxyl termini. Thus, a peptide having the sequence Fmoc-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His-OH (SEQ. ID NO. 1) is obtained and further purified using HPLC.

Alternatively, while the same orthogonal protecting strategy in the synthesis of nesiritide is kept in regards to N-terminus protection and protection of the His residue, there is a consideration of the protecting strategy of Cys residues that should be taken into account. Protection of the Cys residue could be done in two alternative ways: either by acid labile protecting group (such as Trt) or by an orthogonal protecting group that is not cleaved under acidic conditions (such as Acm). If the acid labile protecting group is chosen, then the process proceeds as it was explained above. However if the orthogonal Cys protecting scheme is chosen, then at least one of Cys residues should be protected by an orthogonal protecting group. In such a case the protected peptide on the resin will be as following: Fmoc-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(Y)-Lys(Y)-Val-Leu-Arg(Y)-Arg(Y)-O-Resin (SEQ. ID NO. 1) or Fmoc-Ser(Y)-Pro-Lys(Y)-Met-Val-Gln(Y)-Gly-Ser(Y)-Gly-Cys(Y)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-O-Resin (SEQ. ID NO. 1), wherein U is a thiol containing orthogonal protecting group and Y is an acid-labile protecting group. After coupling with the His residue the following fully protected peptides will be obtained: Fmoc-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(Y)-Lys(Y)-Val-Leu-Arg(Y)-Arg(Y)-His(X)-O(Z) (SEQ. ID NO. 1), or Fmoc-Ser(Y)-Pro-Lys(Y)-Met-Val-Gln(Y)-Gly-Ser(Y)-Gly-Cys(Y)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-His(X)-O(Z) (SEQ. ID NO. 1), wherein X is an acid labile or orthogonal protecting group, Z is a carboxyl-terminal histidine protecting group and U is a thiol containing orthogonal protecting group. After removal of acid labile protecting groups two optional semi-protected peptides could be obtained: Fmoc-Ser-Pro-Lys-Met-Val-Gln-Gly-Ser-Gly-Cys(U)-Phe-Gly-Arg-Lys-Met-Asp-Arg-Ile-Ser-Ser-Ser-Ser-Gly-Leu-Gly-Cys-Lys-Val-Leu-Arg-Arg-His-OH (SEQ. ID NO. 1) or Fmoc-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys(U)-Lys-Val-Leu-Arg-Arg-His-OH (SEQ. ID NO. 1), which is purified using HPLC and then cyclized via the disulfide bond, followed by deprotection of the Fmoc group. Alternatively the purified semi-protected peptide could be first deprotected to remove Fmoc group and then cyclized. The protection group U can either be deprotected prior to the cyclization step using a deprotecting agent or during the cyclization step using iodine. The resulting cyclic peptide is purified by preparative HPLC followed by exchanging its counter-ion to citrate. The peptide may be optionally concentrated in solution. The peptide-citrate solution may be dried by spray-drying or lyophilization to provide a powder.

Suitable co-solvents are solvents that dissolve in the dichloromethane solution but are not suitable for dissolving the protected peptide. Suitable co-solvents may include, but are not limited to, hydrocarbons such as pentane, hexane, or heptane but they could be in some cases (depending on the peptide structure) alcohols, ethers or mixtures thereof.

In another exemplary embodiment demonstrating synthesizing a peptide with a histidine residue at the amino terminal of a peptide, Exenatide (SEQ. ID NO. 2) is produced by providing a fully protected peptide-resin having a formula of Fmoc-His(X or Y)-Gly-Glu(Y)-Gly-Thr(Y)-Phe-Thr(Y)-Ser(Y)-Asp(Y)-Leu-Ser(Y)-Lys(Y)-Gln(Y)-Met-Glu(Y)-Glu(Y)-Glu(Y)-Ala-Val-Arg(Y)-Leu-Phe-Ile-Glu(Y)-Trp(Y)-Leu-Lys(Y)-Asp(Y)-Gly-Gly-Pro-Ser(Y)-Ser(Y)-Gly-Ala-Pro-Pro-Pro-Ser(Y)-Resin (SEQ. ID NO. 2), wherein Fmoc and X are orthogonal protecting groups and Y is an acid-labile protecting group. The fully protected peptide-resin is treated with an acidic composition to produce a semi-protected peptide that is cleaved from the resin, such as Fmoc-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asp-Gly-Gly-Pro-Ser-Ser-Gly-Ala-Pro-Pro-Pro-Ser-NH₂ (SEQ. ID NO. 2) (if the amino terminal histidine is protected with an acid labile protecting group) or Fmoc-His(Fmoc)-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asp-Gly-Gly-Pro-Ser-Ser-Gly-Ala-Pro-Pro-Pro-Ser-NH₂ (SEQ. ID NO. 2) or Fmoc-His(DNP)-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asp-Gly-Gly-Pro-Ser-Ser-Gly-Ala-Pro-Pro-Pro-Ser-NH₂ (SEQ. ID NO. 2) (if the amino terminal histidine is protected with an orthogonal protecting group). The semi-protected peptide is preferably Fmoc-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asp-Gly-Gly-Pro-Ser-Ser-Gly-Ala-Pro-Pro-Pro-Ser-NH₂ (SEQ. ID NO. 2) (i.e., the amino terminal histidine is protected with an acid labile protecting group). The semi-protected peptide is purified by HPLC and then treated with a deprotecting agent such as piperidine to produce the fully deprotected peptide H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asp-Gly-Gly-Pro-Ser-Ser-Gly-Ala-Pro-Pro-Pro-Ser-NH₂ (SEQ. ID NO. 2). The deprotected peptide is then purified by HPLC follow by counterion exchange to acetate. The deprotected peptide may also be optionally concentrated in solution and dried by spray drying or lyophilization to obtain a powder.

In yet another exemplary embodiment demonstrating protecting internal residues with orthogonal protecting groups, Enfuvirtide (SEQ. ID NO. 3) is produced by providing a fully protected peptide-resin having the formula CH₃CO-Tyr(Y)-Thr(Y)-Ser(Y)-Leu-Ile-His(Y)-Ser(Y)-Leu-Ile-Glu(Y)-Glu(Y)-Ser(Y)-Gln(Y)-Asn(Y)-Gln(Y)-Gln(Y)-Glu(Y)-Lys(CBZ)-Asn(Y)-Glu(Y)-Gln(Y)-Glu(Y)-Leu-Leu-Glu(Y)-Leu-Asp(Y)-Lys(CBZ)-Trp(Y)-Ala-Ser(Y)-Leu-Trp(Y)-Asn(Y)-Trp(Y)-Phe-Resin (SEQ. ID NO. 3), wherein CBZ is an orthogonal protecting group and Y is an acid-labile protecting group. The fully protected peptide is treated with an acidic composition that simultaneously cleaves the acid labile protecting groups and the resin to produce the semi-protected peptide CH₃CO-Tyr-Thr-Ser-Leu-Ile-His-Ser-Leu-Ile-Glu-Glu-Ser-Gln-Asn-Gln-Gln-Glu-Lys(CBZ)-Asn-Glu-Gln-Glu-Leu-Leu-Glu-Leu-Asp-Lys(CBZ)-Trp-Ala-Ser-Leu-Trp-Asn-Trp-Phe-NH₂ (SEQ. ID NO. 3). The semi-protected peptide is purified by HPLC. The orthogonal protecting group on the Lys residues may be removed by treating the partially protected peptide with the deprotecting agent HBr/AcOH to produce the peptide CH₃CO-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-leu-Asp-Lys-Trp-Ala-Ser-Leu-Trp-Asn-Trp-Phe-NH₂ (SEQ. ID NO. 3). The resulting fully deprotected peptide is purified with HPLC. The fully deprotected peptide undergoes counterion exchange to acetate. The deprotected peptide may also be optionally concentrated in solution and after that dried by spray drying or lyophilization to obtain a powder.

Having described the invention with reference to certain preferred embodiments, other embodiments will become apparent to one skilled in the art from consideration of the specification. The invention is further defined by reference to the following examples describing in detail the process for purifying peptides. It will be apparent to those skilled in the art that many modifications, both to materials and methods, may be practiced without departing from the scope of the invention.

### EXAMPLES

### Example 1

### Preparation of Nesiritide (SEQ. ID NO. 1) (Fully and Semi-Protected Peptides)

Protected peptide fragment (Fmoc-Ser(tBu)-Pro-Lys(Boc)-Met-Val-Gln(Trt)-Gly-Ser(Trt)-Gly-Cys(Trt)-Phe-Gly-Arg(Pbf)-Lys(Boc)-Met-Asp(OtBu)-Arg(Pbf)-Ile-Ser(Trt)-Ser(Trt)-Ser(Trt)-Ser(Trt)-Gly-Leu-Gly-Cys(Trt)-Lys(Boc)-Val-Leu-Arg(Pbf)-Arg(Pbf)-O-resin)(SEQ. ID NO. 1) was prepared on CTC resin via regular stepwise Fmoc SPPS (solid phase peptide synthesis) procedure starting from loading Fmoc-Arg(Pbf)-OH to CTC resin to obtain substitution of about 0.3 mmol/g. After washing the resin and removing the Fmoc protecting group from the carboxyl terminal amino acid, the second amino acid (Fmoc-Arg(Pbf)) was introduced to start the second coupling step, Fmoc protected amino acids were activated in situ using TBTU/HOBt (N-hydroxybenzotriazole) and subsequently coupled to the resin for 50 minutes. Diisopropylethylamine or collidine were used during coupling as an organic base. Completion of the coupling for each amino acid was indicated by ninhydrine test. After washing the resin, the Fmoc protecting group on the α-amine of the most recently added amino acid was removed with 20% piperidine in DMF for 20 min. These steps were repeated each time with addition of another amino acid according to peptide sequence until the peptide was complete. All amino acids used were Fmoc-N^{α} protected. Trifunctional amino acids were also side chain protected as follows: Ser(tBu), Ser(Trt), Lys(Boc), Gln(Trt), Cys(Trt), Asp(OtBu), Arg(Pbf). Three equivalents of the activated amino acids were used in the coupling reactions. At the end of the synthesis the Fmoc protecting group on the amino terminal Ser was not removed and the peptide-resin was washed with DMF, followed by MeOH, and dried under vacuum to obtain a dry peptide-resin.

The fully protected peptide was cleaved from the resin by washing with a solution of 2% TFA in DCM. TFA was neutralized by DIPEA and equimolar amount of H-His(Trt)-OtBu was added. Condensation between both segments was achieved by addition of TBTU/HOBt solution. The completion of the condensation reaction was monitored by LC/MS. After completion of the reaction the solvent was evaporated and the peptide was treated with an acidic composition (95% TFA, 2.5% TIS, 2.5% EDT). The reaction was continued for 2 hours at room temperature. The product was precipitated by the addition of 10 volumes of MTBE, filtered and dried in vacuum to obtain the semi-protected linear peptide.

### Example 2

### Preparation of Nesiritide (SEQ. ID NO. 1) (Fully Deprotected Peptide)

Fmoc-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His-OH (SEQ. ID NO. 1) (prepared as described in Example 1) was purified on preparative C₁₈ RP-HPLC column. Fractions containing >95% pure product were combined and piperidine was added at a quantity suitable to form about 10% solution by volume. At the end of Fmoc deprotection any excess piperidine was neutralized by cold phosphoric acid to about pH=3. The solution of the linear peptide was diluted to concentrations of about 1 g/L. An equimolar amount of iodine in acetic acid was added under vigorous mixing at room temperature and subsequently excess iodine was neutralized by a small amount of ascorbic acid. The resulting solution was loaded on a HPLC preparative column loaded with RP C-18 resin, 15 μm, and purified using linear gradient of water (0.1% TFA)/acetonitrile (3% to 35% acetonitrile in 60 min) to obtain fractions containing Nesiritide (SEQ. ID NO. 1) trifluoroacetate at a purity of >98.5%. The resulting peptide fractions were treated to replace TFA ions by citrate, collected and lyophilized to obtain final dry peptide (>98.5% pure, [D-His]-Nesiritide (SEQ. ID NO. 1)<0.2%).

The purity of the peptide was determined by analytical HPLC using a Phenomenex® Synergi™ C₁₂ MAX-RP HPLC column. The HPLC column had 4 μm particle size, 80 Å pore size and 250×4.6 mm dimensions. Mobile phase A was 0.05% (v/v) TFA in water, and Mobile phase B was 0.05% (v/v) TFA in ACN. The gradient elution program was from 10% to 25%, and Mobile phase B eluted in 25 minutes. A flow rate of 1 ml/min at 40° C. was used with UV detection at 214 nm. The optical purity (content of D-His) was determined by chiral GC/MS analysis. See Ermer et al., “Quality Control of Peptide Drugs. Chiral Amino Acid Analysis versus Standard for Icatibant Acetate,” Archiv der Pharmazie, 328(9), 635-639 (1995); Gerhardt et al., “Peptides Chemistrie, Structure and Biology,” Proceedings of the 13^{th} American Peptide Symposium, Escom Leidie, p. 241 (1994); Frank et al., “Enantiomer Labeling, A Method for the Quantitative Analysis of Amino Acids,” J. of Chromatography, 167, 187-196 (1978) hereby incorporated by reference.

### Example 3

### Alternative Preparation of Nesiritide (SEQ. ID NO. 1) (Fully and Semi-Protected Peptides)

Protected peptide fragment (Boc-Ser(tBu)-Pro-Lys(Boc)-Met-Val-Gln(Trt)-Gly-Ser(Trt)-Gly-Cys(Trt)-Phe-Gly-Arg(Pbf)-Lys(Boc)-Met-Asp(OtBu)-Arg(Pbf)-Ile-Ser(Trt)-Ser(Trt)-Ser(Trt)-Ser(Trt)-Gly-Leu-Gly-Cys(Trt)-Lys(Boc)-Val-Leu-Arg(Pbf)-Arg(Pbf)-O-resin) (SEQ. ID No. 1) was prepared on CTC resin via regular stepwise Fmoc SPPS (solid phase peptide synthesis) procedure starting from loading Fmoc-Arg(Pbf)-OH to CTC resin to obtain substitution of about 0.3 mmol/g. After washing the resin and removing the Fmoc protecting group from the carboxyl terminal amino acid, the second amino acid (Fmoc-Arg(Pbf)) was introduced to start the second coupling step. Fmoc protected amino acids were activated in situ using TBTU/HOBt (N-hydroxybenzotriazole) and subsequently coupled to the resin for 50 minutes. Diisopropylethylamine or collidine were used during coupling as an organic base. Completion of the coupling for each amino acid was indicated by ninhydrine test. After washing the resin, the Fmoc protecting group on the α-amine of the most recently-added amino acid was removed with 20% piperidine in DMF for 20 min. These steps were repeated each time with addition of another amino acid according to peptide sequence until the peptide was complete. All amino acids used were Fmoc-N^{α} protected except for the last amino acid Boc-Ser(tBu)-OH. Trifunctional amino acids were also side chain protected as follows: Ser(tBu), Ser(Trt), Lys(Boc), Gln(Trt), Cys(Trt), Asp(OtBu), Arg(Pbf). Three equivalents of the activated amino acids were used in the coupling reactions. At the end of the synthesis the peptide-resin was washed with DMF, followed by MeOH, and dried under vacuum to obtain a dry peptide-resin.

The fully protected peptide was cleaved from the resin by washing with a solution of 2% TFA in DCM. TFA was neutralized by DIPEA and equimolar amount of H—His(Trt)-OtBu was added. Condensation between both segments was achieved by addition of TBTU/HOBt solution. The completion of the condensation reaction was monitored by LC/MS. After completion of the reaction the solvent was evaporated and the peptide was treated with an acidic composition (95% TFA, 2.5% TIS, 2.5% EDT). The reaction was continued for 2 hours at room temperature. The product was precipitated by the addition of 10 volumes of MTBE, filtered and dried in vacuum to obtain the fully deprotected linear peptide.

### Example 4

### Alternative Preparation of Nesiritide (SEQ. ID NO. 1) (Fully Deprotected Peptide)

Linear H-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His-OH (SEQ. ID NO. 1) peptide (prepared as described in Example 3) was purified on preparative C₁₈ RP-HPLC column. Fractions containing >95% pure product were combined. The solution of the linear peptide was diluted to concentrations of about 1 g/L. An equimolar amount of iodine in acetic acid was added under vigorous mixing at room temperature and subsequently excess iodine was neutralized by small amount of ascorbic acid. The resulting solution was loaded on a HPLC preparative column loaded with RP C-18 resin, 15 μm, and purified using a linear gradient of water (0.1% TFA)/acetonitrile (3% to 35% acetonitrile in 60 min) to obtain fractions containing Nesiritide (SEQ. ID NO. 1) trifluoroacetate at a purity of >98.5%. The fractions were treated to replace TFA ions by citrate, collected and lyophilized to obtain final dry peptide (>98.5% pure, [D-His]-Nesiritide (SEQ. ID NO. 1)<0.1%).

The purity of the peptide was determined by analytical HPLC using a Phenomenex® Synergi™ C₁₂ MAX-RP HPLC column. The HPLC column had 4 μm particle size, 80 Å pore size and 250×4.6 mm dimensions. Mobile phase A was 0.05% (v/v) TFA in water, and Mobile phase B was 0.05% (v/v) TFA in ACN. The gradient elution program was from 10% to 25%, and Mobile phase B eluted in 25 minutes. A flow rate of 1 ml/min at 40° C. was used with UV detection at 214 nm. The optical purity (content of D-His) was determined by chiral GC/MS analysis.

### Example 5

### Preparation of Teriparatide (Fully and Semi-Protected Peptides)

(SEQ. ID NO.4)

(Fmoc-Ser-Val-Ser-Glu-Ile-Gln-Leu-Met-His-Asn-Leu-
Gly-Lys-His-Leu-Asn-Ser-Met-Glu-Arg-Val-Glu-Trp-
Leu-Arg-Lys-Lys-Leu-Gln-Asp-Val-His-Asn-Phe-OH

Synthesis of the peptide was carried out by a regular stepwise Fmoc SPPS (solid phase peptide synthesis) procedure starting from 2-Cl-Trt-Cl resin (100 g). The first amino acid Fmoc-Phe-OH was loaded on the solid support to obtain substitution of 0.5 mmol/g. After washing the resin and removing the Fmoc protecting group from the carboxyl terminal amino acid, the second amino acid (Fmoc-Asn(Trt)-OH) was introduced to start the second coupling step. Fmoc protected amino acids were activated in situ using TBTU/HOBt (N-hydroxybenzotriazole) and subsequently coupled to the resin for 60 minutes. Diisopropylethylamine or collidine was used during coupling as an organic base. Completion of the coupling for each amino acid was indicated by ninhydrine test. After washing the resin, the Fmoc protecting group on the α-amine of the most recently added amino acid was removed with 20% piperidine in DMF for 20 min. These steps were repeated each time with addition of another amino acid according to peptide sequence until the peptide was complete. All amino acids used were Fmoc-N^{α} protected. Trifunctional amino acids were also side chain protected as follows: Glu(OtBu), Gln(Trt), His(Trt), Asn(Trt), Lys(Boc), Asp(OtBu), Arg(Pbf). Three equivalents of the activated amino acids were used in the coupling reactions. At the end of the synthesis the Fmoc protecting group on the amino terminal Ser was not removed and the peptide-resin was washed with DMF, followed by MeOH, and dried under vacuum to obtain 474 g of a dry peptide-resin.

The fully protected peptide, prepared as described above, was cleaved from the resin together with removal of acid-labile protecting groups using an acidic composition of 95% TFA, 2.5% TIS, 2.5% EDT solution for 2 hours at room temperature. The product was precipitated by the addition of 10 volumes of ether, filtered and dried in vacuum to obtain 191 g crude peptide.

### Example 6

### Preparation of Teriparatide (SEQ. ID NO. 4) Acetate (Fully Deprotected Peptide)

The crude semi-protected peptide, prepared as described in Example 5, was purified on preparative C₁₈ RP-HPLC column. Fractions containing >95% pure product were combined and piperidine was added in an amount suitable to form about 10% solution by volume. After removing the Fmoc protecting group from the amino terminal amino acid, any excess of piperidine was neutralized by addition of cold phosphoric acid. The resulting solution was loaded on a HPLC preparative column with RP C-18 resin, 15 μm, and purified using a linear gradient of water (0.1% TFA)/acetonitrile (3% to 40% acetonitrile in 30 minutes) and purified to obtain fractions containing Teriparatide (SEQ. ID NO. 4) trifluoroacetate at a purity of >97.5%. The fractions were treated to replace TFA ion by acetate, collected and lyophilized to obtain final teriparatide acetate (31 g, >98.5% pure).

The purity of the peptide was determined by analytical HPLC using a Synergi Fusion C₁₈ RP, 80A, 250×4.6 mm, 4 μm; Mobile Phase A: 0.02% TFA in water, Mobile Phase B: ACN; gradient elution program from 15% to 35% B in 20 min; 1 ml/min; 220 nm; 40° C.

The optical purity was determined by chiral GC/MS analysis. See Ermer et al., “Quality Control of Peptide Drugs. Chiral Amino Acid Analysis versus Standard for Icatibant Acetate,” Archiv der Pharmazie, 328(9), 635-639 (1995); Gerhardt et al., “Peptides Chemistrie, Structure and Biology,” Proceedings of the 13^{th} American Peptide Symposium, Escom Leidien, p. 241 (1994); Frank et al., “Enantiomer Labeling, A Method for the Quantitative Analysis of Amino Acids,” J. of Chromatography, 167, 187-196 (1978) hereby incorporated by reference.

### Example 7

### Preparation of Bivalirudin (H-D-Phe-Pro-Arg-Pro-Gly-Glv-Glv-Glv-Asn-Gly-Asp-Phe-Glu-Glu-Ile-Pro-Glu-Glu-Tyr-Leu-OH) (SEQ. ID NO. 6)

Synthesis of the peptide sequence was carried out by stepwise Fmoc SPPS (solid phase peptide synthesis) procedure starting with loading the protected C-terminal amino acid Fmoc-Leu-OH to 2-Cl-Trt-Cl resin. The resin (2-Cl-Trt-Cl resin, 20 g) after washing was stirred with a solution of Fmoc-Leu-OH (17.0 g) in DMF in the presence of diisopropylethylamine for 2 h. After washing the resin the Fmoc protecting group was removed by treatment with 20% piperidine in DMF. After washing of residual reagents the second amino acid (Fmoc-Tyr(tBu)) was introduced to start the first coupling step. The Fmoc protected amino acid was activated in situ using TBTU/HOBt (N-hydroxybenzotriazole) or DIC/OHBt and subsequently coupled to the resin for 50 minutes. Diisopropylethylamine was used during coupling as an organic base. Completion of the coupling for each amino acid was indicated by a Ninhydrine test. After washing the resin, the Fmoc protecting group on the α-amine of the most recently added amino acid was removed with 20% piperidine in DMF for 20 min. These steps were repeated each time with addition of another amino acid according to peptide sequence until the peptide was complete. All amino acids used were Fmoc-N^{α} protected. After addition of the last amino acid in the sequence, Fmoc-D-Phe-OH, Fmoc group was not removed from the peptide-resin. Trifunctional amino acids were also side chain protected as follows: Ser(tBu), Arg(Pbf), Tyr(tBu), Asp(OtBu) and Glu(OtBu). Three equivalents of the activated amino acids were employed in the coupling reactions. At the end of the synthesis the peptide-resin was washed with DMF, followed by MeOH, and dried under vacuum to obtain 62 g dry peptide-resin.

Cleavage of the peptide from the resin and simultaneous deprotection of the acid-labile protecting groups was accomplished by adding the 62 g peptide resin (obtained as described above) to a reactor containing a cold solution of 95% TFA, 2.5% TIS, 2.5% EDT (acidic composition). The mixture was mixed for 2 hours at room temperature, and the product was precipitated by adding 10 volumes of ether (MTBE), then filtered and dried in vacuum to obtain 34.5 g crude product.

The crude semi-protected peptide (34.5 g) obtained above was dissolved in aqueous solution of acetonitrile and loaded on a preparative C₁₈ RP-HPLC column and purified to obtain fractions containing >95% pure product. These fractions were combined and the deprotecting agent piperidine was added in amount suitable to form about a 10% solution by volume. After deblocking the Fmoc group from the amino terminal amino acid, any excess of piperidine was neutralized by addition of cold phosphoric acid. The resulting solution was loaded on a HPLC preparative column loaded with RP C-18 resin, 15 μm, and purified using linear gradient of water (0.1% TFA)/acetonitrile (10% to 15% acetonitrile in 5 minutes and to 38% in 40 min) column and purified to obtain fractions containing Bivalirudin (SEQ. ID NO. 6) at a purity of >97.5%. The counter-ion was exchanged to TFA and pure fractions were collected and lyophilized to obtain a final dry peptide (4.8 g)>99.0% pure (HPLC). It contained not more than 0.5% [Asp⁹-Bivalirudin] (SEQ. ID NO. 6), not more than 0.5% [+Gly]-Bivalirudin (SEQ. ID NO. 6) and not more than 0.5% of any other impurity.

The purity of the peptide was determined by analytical HPLC using a Phenomenex® Synergi™ C₁₂ MAX-RP HPLC column. The HPLC column had 4 μm particle size, 80 Å pore size and 250×4.6 mm dimensions. Mobile phase A was 0.05% (v/v) TFA in water, and Mobile phase B was 0.05% (v/v) TFA in ACN. The gradient elution program was from 17% to 40%, and Mobile phase B eluted in 30 minutes. A flow rate of 1 ml/min at 40° C. was used with UV detection at 214 nm. The optical purity was determined by chiral GC/MS analysis. See Ermer et al., “Quality Control of Peptide Drugs. Chiral Amino Acid Analysis versus Standard for Icatibant Acetate,” Archiv der Pharmazie, 328(9), 635-639 (1995); Gerhardt et al., “Peptides Chemistrie, Structure and Biology,” Proceedings of the 13^{th} American Peptide Symposium, Escom Leidien, p. 241 (1994); Frank et al., “Enantiomer Labeling, A Method for the Quantitative Analysis of Amino Acids,” J. of Chromatography, 167, 187-196 (1978) hereby incorporated by reference.

### Example 8

### Preparation of Exenatide (SEQ. ID NO. 2)

Synthesis of the peptide was carried out by a regular stepwise Fmoc SPPS procedure starting from Rink amide AM resin (100 g). The first amino acid (Fmoc-Ser(tBu)-OH) was loaded on the resin by a regular coupling procedure after removing the Fmoc group from the first amino acid. After washing the resin, the second amino acid (Fmoc-Pro-OH) was introduced to continue sequence elongation. Fmoc protected amino acids were activated in situ using TBTU/HOBt or DIC/HOBt and subsequently coupled to the resin during about 60 minutes. Diisopropylethylamine was used during coupling as an organic base. Completion of the coupling of each amino acid was indicated by a Ninhydrine test. After washing the resin, the Fmoc protecting group on the α-amine of the most recently added amino acid was removed with 20% piperidine in DMF for 20 min. These steps were repeated each time with addition of another amino acid according to peptide sequence until the peptide was complete. All amino acids used were Fmoc-N^{α} protected. After addition of the last amino acid in the sequence (the amino terminal amino acid), Fmoc-His(Trt)-OH, the Fmoc group was not removed from the peptide-resin. Trifunctional amino acids were also side chain protected as follows: His(Trt), Glu(OtBu), Thr(tBu), Ser(tBu), Arg(Pbf), Lys(Boc), Gln(Trt), Asp(OtBu) and Asn(Trt). Three equivalents of the activated amino acids were employed in the coupling reactions. At the end of the synthesis the peptide-resin was washed with DMF, followed by MeOH, and dried under vacuum to obtain dry peptide-resin.

Cleavage of the peptide from the resin and simultaneous deprotection of the acid-labile protecting groups was accomplished by adding the peptide resin obtained as described above to a reactor containing a cold solution of 95% TFA, 2.5% TIS, 2.5% EDT, (acidic composition). The mixture was mixed for 2 hours at room temperature, and the product was precipitated by the addition of 10 volumes of ether (MTBE), then filtered and dried in vacuum to obtain a crude product.

The crude semi-protected peptide product was dissolved in an aqueous solution of acetonitrile. The resulting solution was loaded on a HPLC preparative column loaded with RP C-18 resin, 15 μm, and purified using linear gradient of water (0.1% TFA)/acetonitrile (5% to 15% acetonitrile in 7 minutes and to 40% in 50 min) column and purified to obtain fractions containing >95% pure peptide. These fractions were combined and the deprotecting agent piperidine was added in an amount suitable to form about 10% peptide solution by volume. After deblocking the Fmoc group from the amino terminal amino acid, an excess of piperidine was neutralized by adding cold phosphoric acid. The resulting solution was loaded on a preparative C₁₈ RP-HPLC column and purified to obtain fractions containing Exenatide (SEQ. ID NO. 2) at a purity of >97.5%. The counter-ion was exchanged to acetate and pure fractions were collected and lyophilized to obtain a final dry peptide >98.5% pure (HPLC) and any impurity <0.5%.

The purity of the peptide was determined by analytical HPLC using a Dionex Acclaim Surfactant column. The HPLC column had 5 μm particle size, 120 Å pore size and 250×4.6 mm dimensions. Mobile phase A was 0.05% (v/v) TFA in water, and Mobile phase B was 0.05% (v/v) TFA in ACN. The gradient elution program was from 10% to 30%, and Mobile phase B eluted in 20 minutes. A flow rate of 1 ml/min at 40° C. was used with UV detection at 214 nm. The optical purity was determined by chiral GC/MS analysis. See Ermer et al., “Quality Control of Peptide Drugs. Chiral Amino Acid Analysis versus Standard for Icatibant Acetate,” Archiv der Pharmazie, 328(9), 635-639 (1995); Gerhardt et al., “Peptides Chemistrie, Structure and Biology,” Proceedings of the 13^{th} American Peptide Symposium, Escom Leidien, p. 241 (1994); Frank et al., “Enantiomer Labeling, A Method for the Quantitative Analysis of Amino Acids,” J. of Chromatography, 167, 187-196 (1978) hereby incorporated by reference.

### Example 9

### Preparation of Sermorelin (H-Tyr-Ala-Asp-Ala-Ile-Tyr-Ala-Asp-Ile-Phe-Thr-Asn-Ser-Tyr-Arg-Lys-Val-Leu-Gly-Gln-Leu-Ser-Ala-Arg-Lys-Leu-Leu-Gln-Asp-Ile-Met-Ser-Arg-OH) (SEQ. ID NO. 7)

Synthesis of the peptide sequence is carried out by a stepwise Fmoc SPPS (solid phase peptide synthesis) procedure starting with loading Fmoc-Arg(Pbf)-OH (the carboxyl terminal amino acid) to 2-Cl-Trt-Cl resin. After washing, the resin is treated with a solution of Fmoc-Arg(Pbf)-OH in DMF in the presence of diisopropylethylamine. After washing the resin, the Fmoc protecting group is removed by treatment with 20% piperidine in DMF. After washing the residual reagents from the resin, the second amino acid (Fmoc-Ser(tBu)-OH) is introduced to begin coupling. The Fmoc protected amino acid is activated in situ using TBTU/HOBt (N-hydroxybenzotriazole) or DIC/OHBt and subsequently coupled to the resin for 50 minutes. Diisopropylethylamine is used during coupling as an organic base. Completion of the coupling of each amino acid is indicated by a Ninhydrine test. After washing the resin, the Fmoc protecting group on the α-amine of the most recently added amino acid is removed with 20% piperidine in DMF for 20 min. These steps are repeated each time with addition of another amino acid according to peptide sequence. All amino acids used are Fmoc-N^{α} protected. After addition of the last (amino terminal) amino acid in the sequence, Fmoc-Tyr(tBu)-OH, Fmoc group is not removed from the peptide-resin. Trifunctional amino acids are also side chain protected as follows: Tyr(tBu), Asp(OtBu), Thr(tBu), Asn(Trt), Ser(tBu), Arg(Pbf), Lys(Boc) and Gln(Trt). Three equivalents of the activated amino acids are employed in the coupling reactions. At the end of the synthesis the peptide-resin is washed with DMF, followed by MeOH, and dried under vacuum to obtain dry peptide-resin.

Cleavage of the peptide from the resin and simultaneous deprotection of the protecting groups is accomplished by adding a peptide resin (obtained as described above) to a reactor containing a cold solution of 95% TFA, 2.5% TIS, 2.5% EDT (acidic composition). The mixture is mixed for about 2 hours at room temperature, and the product is precipitated by adding 10 volumes of ether (MTBE), then filtered and dried in vacuum to obtain a crude product.

The crude semi-protected peptide is dissolved in an aqueous solution of acetonitrile. The resulting solution is loaded on a preparative C₁₈ RP-HPLC column and purified to obtain fractions containing >95% pure product. These fractions are combined and the deprotecting agent piperidine is added in an amount suitable to form about 10% solution by volume. After deblocking Fmoc group from the amino terminal amino acid, any excess of piperidine is neutralized adding cold phosphoric acid. The resulting solution is loaded on a preparative C₁₈ RP-HPLC column and purified to obtain fractions containing Sermorelin (SEQ. ID NO. 7) at a purity of >97.5%. The counter-ion is exchanged to acetate and pure fractions are collected and lyophilized to obtain a final dry fully deprotected peptide >98.5% pure (HPLC). It contains not more than 0.5% of any impurity.

### Example 10

### Preparation of Corticorelin (H-Ser-Gln-Glu-Pro-Pro-Ile-Ser-Leu-Asp-Leu-Thr-Phe-His-Leu-Leu-Arg-Glu-Val-Leu-Glu-Met-Thr-Lys-Ala-Asp-Gln-Leu-Ala-Gln-Gln-Ala-His-Ser-Asn-Arg-Lys-Leu-Leu-Asp-Ile-Ala-NH₂) (SEQ. ID NO. 8)

Synthesis of the peptide is carried out by a regular stepwise Fmoc SPPS procedure starting from Rink amide resin. The amine group on the Rink amide resin is protected by an Fmoc group, which is removed prior to loading the first amino acid. The first amino acid (Fmoc-Ala-OH) is loaded on the resin by a regular coupling procedure after removing the Fmoc group from the resin. After washing the resin, the Fmoc protecting group is removed by treatment with 20% piperidine in DMF. After washing residual reagents from the resin, the second amino acid (Fmoc-Ile-OH) is introduced to start the second coupling step. The Fmoc protected amino acid is activated in situ using TBTU/HOBt (N-hydroxybenzotriazole) or DIC/OHBt and subsequently coupled to the resin for 50 minutes. Diisopropylethylamine is used during coupling as an organic base. Completion of the coupling of each amino acid is indicated by a Ninhydrine test. After washing the resin, the Fmoc protecting group on the α-amine of the most recently added amino acid is removed with 20% piperidine in DMF for 20 min. These steps are repeated each time with addition of another amino acid according to peptide sequence until the sequence is complete. All amino acids used are Fmoc-N^{α} protected. After addition of the last amino acid in the sequence, Fmoc-Ser(tBu)-OH, the Fmoc group is not removed from the amino terminal amino acid. Trifunctional amino acids are also side chain protected as follows: Glu(OtBu), Asp(OtBu), Thr(tBu), His(Trt), Ser(tBu), Arg(Pbf), Lys(Boc), Asn(Trt) and Gln(Trt). Three equivalents of the activated amino acids are employed in the coupling reactions. At the end of the synthesis the peptide-resin is washed with DMF, followed by MeOH, and dried under vacuum to obtain dry peptide-resin.

Cleavage of the peptide from the resin and simultaneous deprotection of the acid-labile protecting groups is accomplished by adding a peptide resin (obtained as described above) to a reactor containing a cold solution of 95% TFA, 2.5% TIS, 2.5% EDT (acidic composition). The mixture is mixed for about 2 hours at room temperature, and the product is precipitated by the addition of 10 volumes of ether (MTBE), then filtered and dried in vacuum to obtain a crude product.

The crude semi-protected peptide is dissolved in aqueous solution of acetonitrile. The resulting solution is loaded on a preparative C₁₈ RP-HPLC column and purified to obtain fractions containing >95% pure product. These fractions are combined and the deprotecting agent piperidine is added in an amount suitable to form about 10% solution by volume. After deblocking the Fmoc group from the amino terminal amino acid, any excess of piperidine is neutralized by adding cold phosphoric acid. The resulting solution is loaded on a preparative C₁₈ RP-HPLC column and purified to obtain fractions containing Corticorelin (SEQ. ID NO. 8) at a purity of >97.5%. The counter-ion is exchanged to acetate and pure fractions are collected and lyophilized to obtain a final dry peptide >98.5% pure (HPLC). It contains not more than 0.5% of any impurity.

### Example 11

### Preparation of Thymosin alpha 1 (acetyl-Ser-Asp-Ala-Ala-Val-Asp-Thr-Ser-Ser-Glu-Ile-Thr-Thr-Lys-Asp-Leu-Lys-Glu-Lys-Lys-Glu-Val-Val-Glu-Glu-Ala-Glu-Asn-OH) (SEQ. ID NO. 9)

Synthesis of the peptide sequence is carried out by a stepwise Fmoc SPPS (solid phase peptide synthesis) procedure starting with loading Fmoc-Asn(Trt)-OH to 2-Cl-Trt-Cl resin. After washing, the resin is treated with a solution of Fmoc-Asn(Trt)-OH in DMF in the presence of diisopropylethylamine. After washing the resin, the Fmoc protecting group is removed from the first amino acid by treatment with 20% piperidine in DMF. After washing residual reagents from the resin, the second amino acid (Fmoc-Glu(OtBu)-OH) is introduced to start the first coupling step. The Fmoc protected amino acid is activated in situ using TBTU/HOBt (N-hydroxybenzotriazole) or DIC/OHBt and subsequently coupled to the resin for 50 minutes. Diisopropylethylamine is used during coupling as an organic base. Completion of the coupling for each amino acid is indicated by a Ninhydrine test. After washing the resin to remove excess reagents, the Fmoc protecting group on the α-amine of the most recently added amino acid is removed with 20% piperidine in DMF for 20 min. These steps are repeated each time with addition of another amino acid according to the peptide sequence until the sequence is complete. All amino acids used are Fmoc-N^{α} protected. After addition of the last amino acid in the sequence, Fmoc-Ser(tBu)-OH, the Fmoc group is removed and acetylation of the N-terminal amino group is done. Trifunctional amino acids are also side chain protected as follows: Ser(tBu), Asp(OtBu), Thr(tBu), Glu(OtBu), Lys(CBZ), Lys(Boc) and Asn(Trt). Three equivalents of the activated amino acids are employed in the coupling reactions. At the end of the synthesis the peptide-resin is washed with DMF, followed by MeOH, and dried under vacuum to obtain dry peptide-resin.

Cleavage of the peptide from the resin and simultaneous deprotection of the acid-labile protecting groups is accomplished by adding the peptide resin (obtained as described above) to a reactor containing a cold solution of 95% TFA, 2.5% TIS, 2.5% EDT (acidic composition). The mixture is mixed for about 2 hours at room temperature, and the product is precipitated by the addition of 10 volumes of ether (MTBE), then filtered and dried in vacuum to obtain a crude product.

The crude peptide is dissolved in aqueous solution of acetonitrile. The resulting solution is loaded on a C₁₈ RP-HPLC column and purified to obtain fractions containing >95% pure product. These fractions are combined, concentrated and treated with about 30% (about 10:1 HBr/AC to peptide) to remove the CBZ groups. After deblocking the CBZ group, the peptide is precipitated in MTBE. It is dissolved in aqueous solution of acetonitrile, loaded on a C₁₈ RP-HPLC column and purified to obtain fractions containing Thymosin alpha 1 (SEQ. ID NO. 9) at a purity of >97.5%. The counter-ion is exchanged to acetate and pure fractions are collected and lyophilized to obtain a final dry peptide >98.5% pure (HPLC). It contains not more than 0.5% of any impurity.

### Example 12

### Preparation of Secretin (H-His-Ser-Asp-Gly-Thr-Phe-Thr-Ser-Glu-Leu-Ser-Arg-Leu-Arg-Asp-Ser-Ala-Arg-Leu-Gln-Arg-Leu-Leu-Gln-Gly-Leu-Val-NH₂) (SEQ. ID NO. 10)

Synthesis of the peptide is carried out by a regular stepwise Fmoc SPPS procedure starting from Rink amide resin. The amine group on the Rink amide resin is protected by an Fmoc group, which is removed prior to loading the first amino acid. The first amino acid (Fmoc-Val-OH) is loaded on the resin by a regular coupling procedure after removing of the Fmoc group from the resin. After washing the resin, the Fmoc protecting group is removed from the carboxyl terminal amino acid by treatment with 20% piperidine in DMF. After washing residual reagents from the resin, the second amino acid (Fmoc-Leu-OH) is introduced to start the second coupling step. The Fmoc protected amino acid is activated in situ using TBTU/HOBt (N-hydroxybenzotriazole) or DIC/OHBt and subsequently coupled to the resin for 50 minutes. Diisopropylethylamine is used during coupling as an organic base. Completion of the coupling for each amino acid is indicated by a Ninhydrine test. After washing the resin, the Fmoc protecting group on the α-amine of the most recently added amino acid is removed with 20% piperidine in DMF for 20 min. These steps are repeated each time with another addition of amino acid according to peptide sequence until the sequence is complete. All amino acids used are Fmoc-N^{α} protected. After addition of the last amino acid in the sequence, Fmoc-His(Trt)-OH, the Fmoc group is not removed from the peptide-resin. Trifunctional amino acids are also side chain protected as follows: His(Trt), Ser(tBu), Asp(OtBu), Thr(tBu), Glu(OtBu), Arg(Pbf) and Gln(Trt). Three equivalents of the activated amino acids are employed in the coupling reactions. At the end of the synthesis the peptide-resin is washed with DMF, followed by MeOH, and dried under vacuum to obtain dry peptide-resin.

Cleavage of the peptide from the resin and simultaneous deprotection of the acid-labile protecting groups is accomplished by adding a peptide resin (obtained as described above) to a reactor containing a cold solution of 95% TFA, 2.5% TIS, 2.5% EDT (acidic composition). The mixture is mixed for about 2 hours at room temperature, and the product is precipitated by the addition of 10 volumes of ether (MTBE), then filtered and dried in vacuum to obtain crude product.

The crude semi-protected peptide is dissolved in aqueous solution of acetonitrile. The resulting solution is loaded on a C₁₈ RP-HPLC column and purified to obtain fractions containing >95% pure product. These fractions are combined and piperidine is added in amount suitable to form about 10% solution by volume. After deblocking the Fmoc group on the amino terminal amino acid, any excess of piperidine is neutralized by addition of cold phosphoric acid. The resulting solution is loaded on a C₁₈ RP-HPLC column and purified to obtain fractions containing Secretin (SEQ. ID NO. 10) at a purity of >97.5%. The counter-ion is exchanged to acetate and pure fractions are collected and lyophilized to obtain a final dry peptide >98.5% pure (HPLC). It contains not more than 0.5% of any impurity.

### Example 13

### Preparation of Pramlintide (Fully and Semi-Protected Peptides) (Fmoc-Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Pro-Ile-Leu-Pro-Pro-Thr-Asn-Val-Glv-Ser-Asn-Thr-Tyr-NH₂) (SEQ. ID NO. 11)

Synthesis of the peptide was carried out by a regular stepwise Fmoc SPPS procedure starting from Rink amide AM resin (100 g). The first amino acid (Fmoc-Tyr(tBu)-OH) was loaded on the resin by a regular coupling procedure after removing the Fmoc group from the resin. After washing of the resin to remove excess reagents, the second amino acid (Fmoc-Thr(tBu)) was introduced to continue sequence elongation. Fmoc protected amino acids were activated in situ using TBTU/HOBt or DIC/HOBt and subsequently coupled to the resin during about 60 minutes. Diisopropylethylamine or collidine were used during coupling as an organic base. Completion of the coupling of each amino acid was indicated by ninhydrine test. After washing the resin, the Fmoc protecting group on the α-amine of the most recently added amino acid was removed with 20% piperidine in DMF for 20 min. These steps were repeated each time with addition of another amino acid according to peptide sequence until the sequence was complete. At the end of the addition of the last amino acid (Fmoc-Lys(Boc)-OH) the Fmoc group was not removed. All amino acids used were Fmoc-N^{α} protected. Trifunctional amino acids were also side chain protected as follows: Lys(Boc), Thr(tBu), His(Trt), Ser(tBu), Tyr(tBu), Arg(Pbf), Cys(Trt), Asn(Trt) and Gln(Trt). Three equivalents of the activated amino acids were employed in the coupling reactions. At the end of the synthesis the peptide-resin was washed with DMF, followed by DCM, and dried under vacuum to obtain dry peptide-resin.

The resulting peptide was cleaved from the resin using an acidic composition of a 94% TFA, 1.0% TIS, 2.5% EDT, 2.5% water solution for 1.5 hours at room temperature. The product was precipitated by the addition of 10 volumes of ether, then filtered and dried in vacuum to obtain dry peptide powder. It was identified by LC/MS as Fmoc-Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Pro-Ile-Leu-Pro-Pro-Thr-Asn-Val-Gly-Ser-Asn-Thr-Tyr-NH₂.

### Example 14

### Preparation of Pramlintide (SEQ. ID NO. 11) (Fully Deprotected Peptide)

Fmoc-Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Pro-Ile-Leu-Pro-Pro-Thr-Asn-Val-Gly-Ser-Asn-Thr-Tyr-NH₂ (prepared as described in Example 15) was purified on preparative C₁₈ RP-HPLC column. Fractions containing >95% pure product were combined and piperidine was added in amount suitable to form about 10% solution by volume. After deblocking the Fmoc group on the amino terminal amino acid, any excess of piperidine was neutralized by adding cold phosphoric acid. The resulting solution was loaded on a preparative C₁₈ RP-HPLC column and purified to obtain fractions containing linear Pramlintide (SEQ. ID NO. 11) precursor at a purity of >97.5%. These fractions were diluted to concentrations of about 1 g/L. An equimolar amount of iodine in acetic acid was added under vigorous mixing at room temperature and subsequently excess iodine was neutralized by small amount of ascorbic acid. The resulting solution was loaded on a HPLC preparative column loaded with RP C-18 resin, 15 μm, and purified using linear gradient of water (0.1% TFA)/acetonitrile (3% to 15% acetonitrile in 5 minutes and to 35% in 45 minutes) to obtain fractions containing Pramlintide (SEQ. ID NO. 11) trifluoroacetate at a purity of >98.5%. After exchange of the counterion to acetate the fractions were collected and lyophilized to obtain final dry peptide. The peptide was >98.5% pure (by HPLC) and contained no more than 0.5% of any impurities.

The purity of the peptide was determined by analytical HPLC using a Phenomenex® Synergi™ C₁₂ MAX-RP HPLC column. The HPLC column had 4 μm particle size, 80 Å pore size and 250×4.6 mm dimensions. Mobile phase A was 0.05% (v/v) TFA in water and Mobile phase B was 0.05% (v/v) TFA in ACN. The gradient elution program was from 20% to 40%, and Mobile phase B eluted in 20 minutes. A flow rate of 1 ml/min at 40° C. was used with UV detection at 214 nm. The optical purity was determined by chiral GC/MS analysis. See Ermer et al., “Quality Control of Peptide Drugs. Chiral Amino Acid Analysis versus Standard for Icatibant Acetate,” Archiv der Pharmazie, 328(9), 635-639 (1995); Gerhardt et al., “Peptides Chemistrie, Structure and Biology,” Proceedings of the 13^{th} American Peptide Symposium, Escom Leidien, p. 241 (1994); Frank et al., “Enantiomer Labeling, A Method for the Quantitative Analysis of Amino Acids,” J. of Chromatography, 167, 187-196 (1978) hereby incorporated by reference.

### Example 15

### Preparation of Enfuviritide (CH₃CO-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-Leu-Asp-Lys-Trp-Ala-Ser-Leu-Trp-Asn-Trp-Phe-NH₂ (SEQ. ID NO. 3)

Synthesis of the peptide is carried out by a regular stepwise Fmoc SPPS procedure starting from Rink amide resin. The amine group on the Rink amide resin is protected by an Fmoc group, which is removed prior to loading the first amino acid. The first amino acid (Fmoc-Phe-OH) is loaded on the resin by a regular coupling procedure after removing of the Fmoc group from the resin. After washing the resin, the Fmoc protecting group is removed from the carboxyl terminal amino acid by treatment with 20% piperidine in DMF. After washing residual reagents from the resin, the second amino acid (Fmoc-Trp-OH) is introduced to start the second coupling step. The Fmoc protected amino acid is activated in situ using TBTU/HOBt (N-hydroxybenzotriazole) or DIC/OHBt and subsequently coupled to the resin for 50 minutes. Diisopropylethylamine is used during coupling as an organic base. Completion of the coupling for each amino acid is indicated by a Ninhydrine test. After washing the resin, the Fmoc protecting group on the α-amine of the most recently added amino acid is removed with 20% piperidine in DMF for 20 min. These steps are repeated each time with another addition of amino acid according to peptide sequence until the sequence is complete. All amino acids used are Fmoc-N^{α} protected. After addition of the last amino acid in the sequence, Fmoc-Tyr(tBu)-OH, the Fmoc group is removed from the peptide-resin and the N-terminus amino group is acetylated by reaction with acetic anhydride. Trifunctional amino acids are also side chain protected as follows: His(Trt), Ser(tBu), Asp(OtBu), Thr(tBu), Glu(OtBu), Arg(Pbf) Lys(Cbz), Asn(Trt) and Gln(Trt). Three equivalents of the activated amino acids are employed in the coupling reactions. At the end of the synthesis the peptide-resin is washed with DMF, followed by MeOH, and dried under vacuum to obtain dry peptide-resin.

Cleavage of the peptide from the resin and simultaneous deprotection of the acid-labile protecting groups is accomplished by adding a peptide resin (obtained as described above) to a reactor containing a cold solution of 95% TFA, 2.5% TIS, 2.5% EDT (acidic composition). The mixture is mixed for about 2 hours at room temperature, and the product is precipitated by the addition of 10 volumes of ether (MTBE), then filtered and dried in vacuum to obtain crude product.

The crude semi-protected peptide is dissolved in aqueous solution of acetonitrile. The resulting solution is loaded on a C₁₈ RP-HPLC column and purified to obtain fractions containing >95% pure product. These fractions are combined and piperidine is added in amount suitable to form about 10% solution by volume. After deblocking the Fmoc group on the amino terminal amino acid, any excess of piperidine is neutralized by addition of cold phosphoric acid. The resulting solution is loaded on a C₁₈ RP-HPLC column and purified to obtain fractions containing Enfuviritide (SEQ. ID NO. 3) at a purity of >97.5%. The counter-ion is exchanged to acetate and pure fractions are collected and lyophilized to obtain a final dry peptide >98.5% pure (HPLC). It contains not more than 0.5% of any impurity.

We claim:

1. A process for preparing a peptide of high purity comprising: (a) providing a fully protected peptide having at least one acid labile protecting group and at least one orthogonal protecting group; (b) deprotecting the acid labile protecting groups from the fully protected peptide with an acidic composition yielding a semi-protected peptide; (c) purifying the semi-protected peptide by HPLC; (d) deprotecting the orthogonal protecting groups from the semi-protected peptide with a deprotecting agent yielding a fully deprotected peptide; and (e) purifying the fully deprotected peptide by HPLC, wherein if the fully protected peptide contains two or more thiol-containing residues, then all the thiol-containing residues are protected by the acid labile protecting groups.

2. The process of claim 1, wherein the peptide is linear.

3. The process of claim 1, wherein the peptide is cyclic.

4. The process of claim 1, wherein the fully protected peptide is prepared on a solid support or in solution.

5. The process of claim 1, wherein the acid labile protecting group is t-butyl ester, trityl, t-butyloxycarbonyl, 2,2,4,6,7-pentamethyldihydrobenzofuran-5-sulfonyl, or t-butyl.

6. The process of claim 1, wherein the orthogonal protecting group is 9-fluorenylmethyloxycarbonyl, benzyloxycarbonyl, benzyl, α,α-dimethyl-3,5-dimethoxybenzyloxylcarbonyl, 1-(4,4-dimethyl-2,6-dioxocyclohex-1-ylidene)ethyl, 2,4-dinitrophenyl, benzyl ester, 9-fluorenymethyl ester, N^{α}-2-(4-Nitrophenylsulfonyl)ethoxycarbonyl, allyloxycarbonyl or acetamidomethyl.

7. The process of claim 1, wherein the acidic composition comprises an acidic material and at least one scavenger reagent.

8. The process of claim 7, wherein the acidic material is trifluoroacetic acid, hydrofluoric acid, trifluoromethanesulfonic acid or hydrobromic acid/acetic acid.

9. The process of claim 7, wherein the scavenger reagents are triisopropylsilane, 1,2-ethanedithiol, water, phenol, m-cresol, thioanisole, dodecylmercaptane or dithioerythritol.

10. The process of claim 7, wherein the acidic material is present in an amount of about 50% to about 99% by volume of the acidic composition, and the scavenger reagents are present in an amount of about 1% to about 50% by volume of the acidic composition.

11. The process of claim 1, wherein the deprotecting agent is piperidine, 1,8-diazobicyclo[5.4.0]undec-7-ene, p-dimethylaminopyridine, triethylamine, hydrogen bromide/acetic acid, H₂/Pd/C, hydrazine, hydrofluoric acid, or trifluoromethanesulfonic acid.

12. The process of claim 1, wherein the process further comprises at least one step of neutralizing excess deprotecting agent, drying the fully deprotected peptide, purifying the fully deprotected peptide by counter-ion exchange, adding a histidine residue onto a carboxyl terminal of the fully protected peptide or cyclizing the fully deprotected peptide.

13. The process of claim 2, wherein the linear peptide is Elcatonin (SEQ. ID NO. 5), Teriparatide (SEQ. ID NO. 4), Bivalirudin (SEQ. ID NO. 6), Exenatide (SEQ. ID NO. 2), Sermorelin (SEQ. ID NO. 7), Corticorelin (SEQ. ID NO. 8), Enfuvirtide (SEQ. ID NO. 3), Thymosin alpha 1 (SEQ. ID NO. 9), or Secretin (SEQ. ID NO. 10).

14. The process of claim 3, wherein the cyclic peptide is Nesiritide (SEQ. ID NO. 1) or Pramlintide (SEQ. ID NO. 11).

15. The process of claims 13 or 14, wherein the peptide has a purity of at least 97.5% as measured by HPLC.

16. The process of claims 13 or 14, wherein the peptide has a purity of at least 98.5% as measured by HPLC.

17. The process of claims 13 or 14, wherein the peptide has a purity of at least 99% as measured by HPLC.

18. A process for preparing Nesiritide (SEQ. ID NO. 1) comprising: (a) providing a fully protected peptide attached to a highly acid sensitive resin having the formula X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-O-Resin (SEQ. ID NO. 1), wherein X is an acid labile or orthogonal protecting group, U is an acid labile or orthogonal protecting group on a cysteine residue, and Y is an acid labile protecting group; (b) reacting the fully protected peptide with a weak acidic composition to cleave the fully protected peptide from the resin, providing the fully protected peptide X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-OH (SEQ. ID NO. 1) in solution; (c) coupling H-His(X)-O(Z) to produce X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-His(X)-O(Z) (SEQ. ID NO. 1), wherein Z is a carboxy-terminal histidine protecting group which is either an orthogonal or acid-labile protecting group; (d) isolating the fully protected peptide by either evaporating the solvent or precipitating using a suitable co-solvent; (e) deprotecting the acid labile protecting groups in the fully protected peptide by treatment with an acidic composition to produce a semi-protected peptide, X-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His(X)-OZ (SEQ. ID NO. 1) or a non-protected linear peptide H-Ser-Pro-Lys-Met-Val-Gln-Gly-Ser-Gly-Cys-Phe-Gly-Arg-Lys-Met-Asp-Arg-Ile-Ser-Ser-Ser-Ser-Gly-Leu-Gly-Cys-Lys-Val-Leu-Arg-Arg-His-OH (SEQ. ID NO. 1); (f) purifying the semi-protected peptide by preparative HPLC; (g) deprotecting the remaining orthogonal protecting groups from the semi-protected peptide to provide a fully deprotected peptide; (h) purifying the fully deprotected peptide by preparative HPLC; (i) cyclizing the fully deprotected peptide to provide a cyclic peptide; (j) purifying the cyclic peptide by preparative HPLC; (k) exchanging the counter ion of the cyclic peptide to citrate; and (1) drying the cyclic peptide to provide Nesiritide (SEQ. ID NO. 1) in solid powder.

19. Nesiritide (SEQ. ID NO. 1) obtained according to the process of claim 18 containing about 0.05% to about 0.5% [D-His]-Nesiritide (SEQ. ID NO. 1) as determined by chiral GC/MS.

20. The process of claim 18, wherein the highly acid sensitive resin is 2-chlorotrityl-chloride.

21. The process of claim 18, wherein the orthogonal protecting group remained on the semi-protected cysteine residue can be deprotected from the peptide during the cyclization step using iodine.

22. A peptide-resin conjugate of formula 1:
A-B-Resin
wherein A=X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-O-, X is an orthogonal or acid labile protecting group, U is an orthogonal or acid labile protecting group on a cysteine residue, and Y is an acid labile protecting group; B is 2-chlorotrityl-chloride, and Resin is a solid matrix attached to the peptide.

23. The peptide-resin conjugate of claim 22, wherein the protecting group (X) is 9-fluorenylmethyloxycarbonyl or t-butyloxycarbonyl and the protecting group (U) is acetamidomethyl or trityl.

24. The peptide-resin conjugate of claim 23, wherein the protecting group (X) is t-butyloxycarbonyl and the protecting group (U) is trityl.

25. Nesiritide (SEQ. ID NO. 1) containing about 0.05% to about 0.5% [D-His]-Nesiritide (SEQ. ID NO. 1) as determined by chiral GC/MS.

26. Nesiritide (SEQ. ID NO. 1) containing about 0.05% to about 0.5% [D-His]-Nesiritide (SEQ. ID NO. 1) as determined by chiral GC/MS, made by a process comprising; providing X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-OH (SEQ. ID NO. 1) attach to a resin; and coupling of H-His(X)-O(Z) to X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-OH (SEQ. ID NO. 1) after cleavage of X-Ser(Y)-Pro-Lys(Y)-Met-Val-Gln(Y)-Gly-Ser(Y)-Gly-Cys(U)-Phe-Gly-Arg(Y)-Lys(Y)-Met-Asp(Y)-Arg(Y)-Ile-Ser(Y)-Ser(Y)-Ser(Y)-Ser(Y)-Gly-Leu-Gly-Cys(U)-Lys(Y)-Val-Leu-Arg(Y)-Arg(Y)-OH (SEQ. ID NO. 1) from the resin, wherein X is an orthogonal or acid labile protecting group, U is an orthogonal or acid labile protecting group on a cysteine residue, Y is an acid labile protecting group and Z is a carboxy-terminal histidine protecting group which is either an orthogonal or acid labile protecting group.

27. Teriparatide (SEQ. ID NO. 4) having a purity of at least 98.5% as measured by HPLC.

28. Bivalirudin (SEQ. ID NO. 6) containing not more than 0.5% [Asp⁹-Bivalirudin] (SEQ. ID NO. 6), not more than 0.5% [+Gly]-Bivalirudin (SEQ. ID NO. 6) and not more than 0.5% of any other impurity.

29. Exenatide (SEQ. ID NO. 2) having a purity of at least 98.5% as measured by HPLC.

30. Sermorelin (SEQ. ID NO. 7) having a purity of at least 98.5% as measured by HPLC.

31. Corticorelin (SEQ. ID NO. 8) having a purity of at least 98.5% as measured by HPLC.

32. Enfuvirtide (SEQ. ID NO. 3) having a purity of at least 98.5% as measured by HPLC.

33. Thymosin alpha 1 (SEQ. ID NO. 9) having a purity of at least 98.5% as measured by HPLC.

34. Pramlintide (SEQ. ID NO. 11) having a purity of at least 98.5% as measured by HPLC.
